(19) United States
(12) Patent Application Publication
(10) Pub. No.: US 2023/0381062 A1
(43) Pub. Date: Nov. 30, 2023

(54) DEVICE FOR CARDIOPULMONARY RESUSCITATION, PAD, AND METHOD FOR CONTROLLING A DEVICE FOR CARDIOPULMONARY RESUSCITATION

(71) Applicant: WEINMANN EMERGENCY MEDICAL TECHNOLOGY GMBH + CO. KG, Hamburg (DE)

(72) Inventors: Christian NEUHAUS, Quickborn (DE); Tobias KALWA, Hamburg (DE)

(21) Appl. No.: 18/032,840

(22) PCT Filed: Oct. 15, 2021
(86) PCT No.: PCT/DE2021/100831

§ 371 Date: Apr. 20, 2023

(30) Foreign Application Priority Data
Oct. 23, 2020 (DE) 10 2020 127 948.5

(51) Int. Cl. A61H 31/00

(52) U.S. Cl. CPC A61H 31/006; A61H 31/005; A61H 2201/5005; A61H 2201/107; A61H 2201/0188; A61H 2201/5048; A61H 2201/5007; A61H 2201/5061

(57) ABSTRACT

A device for cardiopulmonary resuscitation, a pad for assisting with cardiopulmonary resuscitation, and a method for controlling such a device, wherein the parameters of compression depth and frequency of the heart massage, and at least one vital parameter, are continuously monitored, and the parameters of the heart massage within the context of the position of the action of force on the patient's thorax, the direction of the action of force on the patient's thorax, the compression depth and/or the frequency of the heart massage are optimized on the basis of the individual anatomy of the patient.

The invention relates to an apparatus for cardiopulmonary resuscitation.

The invention also relates to a pad for use within the scope of a cardiopulmonary resuscitation.

Moreover, the invention relates to a method for controlling an apparatus for cardiopulmonary resuscitation, within the scope of which an aider is instructed to carry out a cardiopulmonary resuscitation and/or a thorax compression device is controlled for machine assistance with a cardiopulmonary resuscitation.

Cardiopulmonary resuscitation, abbreviated CPR, is a resuscitation measure which is applied in the case of respiratory and circulatory arrest and which serves to maintain the oxygen supply to the organs, especially the brain and the heart.

Various factors are important when carrying out corresponding resuscitations. In particular, these are the frequency and the compression depth of the cardiac massage. Moreover, active phases and rest phases may be important. In combination with the use of defibrillators, there are likewise different variants regarding the time duration of defibrillation, the number of defibrillations and the length of the pauses between such actions.

The fundamental rules for carrying out corresponding resuscitations are obtained from statistical information relating to large demographic groups. Experimental studies and calculations have derived how a cardiac massage should be carried out for, on average, an optimal result within the scope of a cardiopulmonary resuscitation. Thus, guidelines were drafted, which specify that a cardiac massage should be carried out perpendicularly from above, on the lower half of the sternum, with a compression frequency of 100-120 1/min and a compression depth of 5-6 cm.

In the past, it was found that the corresponding resuscitation measures were carried out with very different success rates for the individual patients. The reason for this is that each individual human has, to a certain extent, an individual anatomy, with the result that the specifications for a cardiac massage or a cardiopulmonary resuscitation with on average an optimal effect cannot achieve equally good results for each individual patient.

In addition to the manual cardiac massage by a human aider, it is also in principle possible to have cardiac massage or cardiopulmonary resuscitation assisted by technology.

The prior art has disclosed apparatuses usable for an automated cardiopulmonary resuscitation. Such apparatuses comprise a thorax compression device which automatically exerts an alternating force on the thorax of a patient, with the result that a cardiac massage is implementable.

Moreover, aids for manual compression of a thorax, for example in the form of pads, are also known.

Moreover, apparatuses which assist a human aider, for example a paramedic, with carrying out a cardiopulmonary resuscitation by way of the provision of information in relation to the implemented cardiopulmonary resuscitation are also known.

The acquisition and preparation of measured data in relation to the cardiac massage as such and/or in relation to the vital parameters of the patient are required as a basis for a functioning open-loop or closed-loop control of the cardiac massage, both in the case of the technical assistance of a human aider and in the case of a fully or partially automated cardiac massage or cardiopulmonary resuscitation.

It is possible to measure the compression frequency and compression depth parameters of the cardiac massage on the basis of, for example, the data acquirable by way of acceleration sensors and/or gyroscope sensors.

The vital parameters of a patient, for example given by the blood pressure and/or the oxygen saturation of the blood, can be measured with the aid of known methods such as cerebral oximetry, pulse oximetry and the known methods for blood pressure measurement.

The generation of instructions or feedback for a human aider or the control of an automated cardiac massage or cardiopulmonary resuscitation on the basis of the compression depth and compression frequency data captured with the aid of suitable measuring means is already known as a matter of principle.

It is an object of the invention to specify an apparatus for cardiopulmonary resuscitation, the said apparatus increasing the probability of obtaining better results for an individual patient.

According to the invention, this object is achieved by an apparatus for cardiopulmonary resuscitation as claimed in patent claim 1.

A further object of the invention lies in specifying a pad for use within the scope of cardiopulmonary resuscitation, the said pad increasing the probability of obtaining better results for an individual patient.

According to the invention, this object is achieved by a pad as claimed in patent claim 7.

A further object of the invention lies in specifying a method for controlling an apparatus for cardiopulmonary resuscitation, the said method increasing the probability of obtaining better results for an individual patient.

According to the invention, this object is achieved by a method for controlling an apparatus for cardiopulmonary resuscitation as claimed in patent claim 10.

The dependent claims specify advantageous embodiments of the invention.

The teaching according to the invention is based on the approach that the optimal position and direction of the action of force on the thorax of a patient during a cardiac massage within the scope of a cardiopulmonary resuscitation varies on account of the anatomy of humans, which is individual to a certain extent.

To obtain the best possible effect of the cardiac massage, the apparatus according to the invention and the method according to the invention for controlling the apparatus are designed so that a variation in the position and/or the direction of the action of force on the thorax of the patient and the continuous monitoring of at least one vital parameter of the patient while the cardiac massage is carried out allow conclusions to be drawn for the individual patient about the optimal position and direction of the action of force on the thorax of the patient, with the result that these conclusions are usable for the cardiac massage of the patient going forwards.

In a further aspect of the invention, inter alia to unburden an aider implementing the cardiac massage, the compression depth and the frequency of the cardiac massage are able to be reduced to such an extent starting from the guidelines that there just is no corresponding deterioration in the vital parameters of the patients.

The features of an apparatus for cardiopulmonary resuscitation disclosed below are a part of the invention, both individually and in all implementable combinations.

An apparatus for cardiopulmonary resuscitation according to the invention is designed at least to assist a cardiopulmonary resuscitation of a patient.

To this end, an apparatus according to the invention for cardiopulmonary resuscitation comprises at least one computing unit, at least one sensor interface, and at least one memory.

The at least one sensor interface is designed to establish a connection to at least one sensor for acquiring the data from a cardiac massage within the meaning of compression depth and compression frequency and to at least one sensor for acquiring at least one vital parameter of a patient.

In an embodiment of the invention, the at least one sensor interface is designed to establish a connection to in each case at least one sensor for acquiring the blood pressure and/or the oxygen saturation in the blood of the patient and at least one sensor for acquiring the compression depth and/or the compression frequency of the cardiac massage.

In an embodiment of the invention, the latter comprises a pad which has at least one sensor for determining the compression depth and/or the frequency of the cardiac massage. The pad is positionable on the chest of the patient and is connectable to the sensor interface.

In an embodiment of the invention, the pad comprises at least one acceleration sensor and/or gyroscope sensor and/or magnetic field sensor with a total of 3, 6 or 9 axes (for example, a 3-axis acceleration sensor and 3-axis gyroscope sensor and 3-axis magnetic field sensor).

The pad comprises a multi-axis acceleration sensor in an embodiment of the invention.

The pad comprises a 6-axis acceleration/angular rate sensor in a preferred embodiment of the invention.

The pad is embodied as a flexible mat in an embodiment of the invention, the said mat being positionable on the chest of the patient.

The pad is able to be adhesively bonded to the chest of the patient in a preferred embodiment of the invention.

The pad comprises a solid housing in a further embodiment of the invention, the at least one sensor for acquiring the compression depth and/or frequency of the cardiac massage being integrated in the said housing.

In an embodiment of the invention, the pad comprises a force sensor for acquiring a force acting on the pad.

In a further embodiment of the invention, the at least one sensor interface is designed to establish a connection not only to the aforementioned sensors but also to at least one sensor for monitoring an at least partially automated ventilation of a patient.

The data transmitted via the sensor interface to the at least one computing unit of the apparatus according to the invention for cardiopulmonary resuscitation are able to be evaluated with the aid of the computing unit.

In an embodiment of the invention, the apparatus for cardiopulmonary resuscitation comprises at least one output apparatus, by means of which it is possible to output information for a human aider.

In this case, the at least one output apparatus can preferably be in the form of a display, an individual illuminant or a plurality of illuminants, and/or in the form of an acoustic output apparatus.

In an embodiment of the invention, the apparatus for cardiopulmonary resuscitation is furthermore designed so that the cardiac massage on a patient is implementable in automated fashion.

To this end, the apparatus comprises a thorax compression device, by means of which the thorax of the patient is compressible for the purposes of carrying out a cardiac massage.

In these embodiments of the invention, the apparatus for cardiopulmonary resuscitation comprises at least one control unit designed to control the automated cardiac massage with the aid of the thorax compression device.

The control signals generable with the aid of the control unit are designed to control the compression frequency, the compression depth, the compression direction, and/or the position of the action of force on the thorax of the patient by the thorax compression device.

In an embodiment of the invention, a thorax compression device comprises a compression head which is advanceable and retractable along an axis. From a rest position, the compression head is movable approximately in the direction of the spinal column in accordance with a preset and/or adjustable length value from the region of the sternum on the upper side of the thorax of a patient.

In an embodiment of the invention, the axis along which the compression head is movable is inclinable, such that the compression of the thorax is made possible not only by a force exerted approximately perpendicularly on the thorax, but also in an angular range about the perpendicularly aligned axis.

In an embodiment of the invention, the movement axis of the compression head is inclinable through an angular range from −15° to 15° about the movement axis which is aligned perpendicular to the thorax.

In a further embodiment of the invention, the movement axis of the compression head is inclinable through an angular range from −10° to 10° about the movement axis which is aligned perpendicular to the thorax.

The position of the compression head on the thorax of a patient is manually changeable by a human aider in an embodiment of the invention.

In another embodiment of the invention, the position of the compression head on the thorax of a patient is automatically changeable, either in addition to the manual change or on an exclusive basis.

To this end, an apparatus according to the invention for cardiopulmonary resuscitation comprises actuators, by means of which the position of the compression head on the thorax of a patient is rendered changeable in at least two dimensions.

In a preferred embodiment of the invention with an at least partially automated thorax compression device for carrying out a cardiac massage, the movement axis of the compression head is inclinable through an angular range about a movement axis which is aligned perpendicular to the thorax and the position of the compression head on the thorax of a patient is automatically changeable, either in addition to the manual change or on an exclusive basis.

In an embodiment of the invention, the at least one output device is designed to output instructions for a human aider.

In the case of a manual cardiac massage by the human aider, optical and/or acoustic instructions regarding the adjustment of the compression frequency, the compression depth, the position and/or the direction of the action of force on the thorax of the patient by the hand of the aider or a pad on the thorax of the patient are able to be output with the aid of the at least one output device.

An optical output of the instructions using arrows and/or pictograms is advantageous here for a quick comprehension of the instructions by a human aider.

In an embodiment of the invention, the output unit is designed to output to a human aider instructions regarding the positioning of the ball of the hand of the human aider that is used for the cardiac massage or the positioning of an optionally used pad.

In an embodiment of the invention, the instructions regarding the positioning of the ball of the hand of the human aider that is used for the cardiac massage or the positioning of an optionally used pad are able to be output optically and/or acoustically with the aid of the output unit.

In a further embodiment of the invention, the apparatus for cardiopulmonary resuscitation is furthermore designed so that instructions regarding the adjustment of the compression depth and/or frequency are able to be output to a human aider with the aid of the output unit, in such a way that the said compression depth and/or frequency are reducible to such an extent starting from the guidelines that there is no deterioration of the vital parameters of the patient while the human aider is unburdened.

In an embodiment of the invention with a thorax compression device, the control unit is designed so that the latter can be used to transmit control signals for changing the position of the compression head on the chest of a patient and/or for changing the movement axis of the compression head to the thorax compression device and/or for adjusting the compression depth and/or the frequency of the cardiac massage.

In an embodiment of the invention, the computing unit is designed so that the data from the sensors regarding the vital parameters of the patient which are acquirable with the aid of the sensor interface are able to be evaluated to the effect of whether the state of the patient in relation to the acquired vital parameters improves or deteriorates over time within the scope of a cardiac massage or cardiopulmonary resuscitation.

In a preferred embodiment of the invention, the apparatus for cardiopulmonary resuscitation is designed to monitor the oxygen saturation in the blood of the patient and/or the blood pressure of the patient and/or the volumetric flow rate of the blood in at least one artery of the patient, for example in a carotid artery.

In an embodiment of the invention, the sensor interface, the computing unit and the output unit, and/or the control unit for controlling a thorax compression device are integrated in a defibrillator.

The features of a method for controlling an apparatus for cardiopulmonary resuscitation disclosed below are a part of the invention, both individually and in all implementable combinations.

In a method according to the invention for controlling an apparatus for cardiopulmonary resuscitation, at least one vital parameter of a patient is acquired in at least one method step, the compression depth and/or the frequency of the cardiac massage applied to the patient are acquired in at least one method step, the acquired data of the at least one vital parameter and of the compression depth and/or frequency of the cardiac massage are stored in a memory, the stored measured data of the at least one vital parameter of previous measurement points are compared in at least one method step with the measured data of the at least one vital parameter acquired in a current method step, the comparison of the stored measured data of the at least one vital parameter of previous measurement points with the measured data of the at least one vital parameter acquired in a current method step is used in at least one method step to ascertain whether there has been an improvement or deterioration or no change in relation to the at least one vital parameter, and a measure for adjusting the position of the action of force on the thorax of the patient and/or for adjusting the angle of the action of force on the thorax of the patient and/or for adjusting the compression depth and/or the frequency of the cardiac massage is determined in a further method step, with the adjustment optionally being able to be 0. In a further method step of a method according to the invention for controlling an apparatus for cardiopulmonary resuscitation, the determined measure is optically and/or acoustically output as an instruction to a human aider with the aid of an output unit and/or converted with the aid of a control unit into appropriate control signals for controlling a thorax compression device.

In an embodiment of the method according to the invention, the thorax compression device is controlled in such a way with the aid of the control signals that the position of the action of force on the thorax of the patient and/or the angle of the action of force on the thorax of the patient and/or the compression depth and/or the frequency of the cardiac massage is adjusted in accordance with the determined measure.

In a method according to the invention, the at least one vital parameter is acquired continuously, at least following the implementation of a previously determined measure, and is compared with the stored measured data of the at least one vital parameter. In accordance with the method steps disclosed above, the comparison of the current with the stored measured data is used to ascertain the success of the previously determined measure, within the meaning of influencing the at least one acquired vital parameter, and a further measure for adjusting the position of the action of force on the thorax of the patient and/or the angle of the action of force on the thorax of the patient and/or the compression depth and/or the frequency of the cardiac massage is optionally determined.

In embodiments of the invention, the value ranges/threshold values listed below are used to determine the success of a measure in relation to a determined vital parameter in each case. For the blood pressure, +/−3 mmHg counts as unchanged, lower is worse, higher is better. For oxygenation, +/−1 vol. % counts as unchanged, lower is worse, higher is better. This applies to both the rSO2 (regional cerebral oxygen saturation) and the PaO2 values (partial arterial oxygen pressure).

A more differentiated assessment is preferably carried out for the respiratory CO2: in this case, a fast increase (+1 vol. %) is good, and an equal drop is bad. However, a slow drop provides no meaningful statement (relative change).

In an embodiment of a method according to the invention, the measure to be output and/or implemented in at least one subsequent method step is determined on the basis of a defined optimization process.

In an embodiment of the invention, the optimization process is designed as a component-by-component optimization of the individual parameters of a cardiac massage.

In an embodiment of the method according to the invention, the direction components of the position of the action of force on the thorax of the patient each are the subject of an individual optimization in this case.

Proceeding from a starting point, the left/right direction component for displacing the position of the action of force on the thorax of the patient from a starting point is in a direction approximately perpendicular to the longitudinal direction of the sternum on the thorax of the patient and the superior/inferior direction component for displacing the position of the action of force on the thorax of the patient is approximately in the longitudinal direction of the sternum in this case, with inferior meaning a displacement in the direction of the lower abdomen and superior meaning a displacement in the direction of the head of the patient.

In an embodiment of the method according to the invention, the starting point of the optimization corresponds to the region on the lower half of the sternum of the patient as defined by the guidelines.

In this context, the term “point” or “starting point” is used, for instance, within the meaning of the center of the region in which the force for carrying out the cardiac massage acts on the thorax of the patient.

In an embodiment of the method according to the invention, the components of the direction of the action of force on the thorax of the patient each are the subject of an individual optimization.

Proceeding from a starting angle, the direction vector of the action of force on the thorax of the patient is varied here as the determined measure.

In an embodiment of the invention, the starting angle of the direction vector of the action of force on the thorax of the patient is defined as 0° within the meaning of this document, with this meaning an approximately perpendicular action of force on the thorax of the patient within the meaning of the guidelines.

The direction vector of the action of force on the thorax of the patient is divided into a sagittal and a transversal component in an embodiment of the invention. In this case, the tip of the direction vector is approximately in the center of the region of the action of force, with a change in the sagittal component corresponding to an inclination of the direction vector approximately in the direction of the head (superior) or lower abdomen (inferior) of the patient and the change in the transversal component directed perpendicular to the sagittal component corresponding to an inclination of the direction vector approximately in the direction of the left or right arm of the patient.

The start of a method according to the invention is implemented by the output of predetermined starting parameters as instructions to a human aider and/or a corresponding control of a thorax compression device.

The starting parameters which are given by the position of the action of force on the thorax of the patient and/or the direction of the action of force on the thorax of the patient and/or the compression depth of the cardiac massage and/or the frequency of the cardiac massage are defined as the values specified by the current guidelines in a preferred embodiment of the invention.

For the currently valid guidelines, the starting parameters are given, as already described above, by the lower half of the sternum as a starting position of the action of force on the thorax of the patient, approximately perpendicular with respect to the sternum as the starting direction of the action of force on the thorax of the patient, approximately 5 to 6 cm as the starting compression depth of the cardiac massage, and approximately 100 to 120 1/min as the starting frequency of the cardiac massage.

In a preferred embodiment of a method according to the invention, each output of a change of a parameter within the scope of a determined measure as an instruction to a human aider and/or the corresponding control of the thorax compression device is followed by an ascertainment of the success of the previously determined measure on the basis of the comparison of the at least one subsequently acquired vital parameter with the stored previous measured data.

In a particularly preferred embodiment, an optionally subsequent measure is determined taking account of the success of the preceding measure.

In an embodiment of the method according to the invention, at least the position of the action of force on the thorax of the patient is optimized along a search path.

The search path initially has at least one starting point within the meaning of the position of the action of force on the thorax of the patient and at least one defined first measure within the meaning of a change in the position of the action of force on the thorax of the patient.

In a preferred embodiment of the method according to the invention with an optimization of at least the position of the action of force on the thorax of the patient along a search path, the search path is completely predetermined and is at least partially read from the memory of the utilized apparatus for cardiopulmonary resuscitation for the purposes of determining the subsequent measures.

In an embodiment of the invention, the search path comprises a plurality of points which each correspond to a position on the thorax of the patient. In this case, the position of the action of force is successively displaced to these points on the search path for the purposes of optimizing at least the position of the action of force on the thorax of the patient, in each case within the meaning of a measure, the respective success of the measure is ascertained and the point corresponding to the position with the greatest ascertained success is ascertained following the traversal of the search path.

In an embodiment of the method according to the invention, the search path is continuously adapted during the optimization of the position of the action of force on the thorax of the patient, the adaptation being based on the success of the preceding measure ascertained with the aid of the acquired vital parameters.

In an embodiment of the invention, an optimization in relation to the direction of the action of force on the thorax of the patient is additionally implemented along the search path.

A pad according to the invention for use during a cardiopulmonary resuscitation comprises a guide device which defines a search path, with a compression of the thorax of a patient being implemented along the search path within the scope of a cardiopulmonary resuscitation.

In an embodiment of the invention, the guide device of the pad is realized by an optical marking on the pad and/or a haptic design of the surface of the pad.

As a result, a human aider can reliably follow the search path with their hand or an auxiliary means, and optionally reliably implement the instructions output with the aid of the apparatus for cardiopulmonary resuscitation.

The haptic design of the surface of the pad as a guide device is configured in such a way in an embodiment of the invention that the search path has a smooth surface and the remaining part of the surface of the pad has a rough embodiment. A reversed design can also realize the desired effect.

In another embodiment of the invention, the surface of the pad along the search path is offset vis-à-vis the remaining surface in such a way that the said surface of the pad along the search path projects from the remaining surface or has a lower height than the latter.

In further embodiments of the invention, the guide device is realized by a guide rail which extends along the search path and in which a handle is displaceably mounted, with a vertical action of force on the handle at the respective position along the search path being transferred to the thorax of the patient.

In advantageous embodiments of the invention, the pad comprises one or more sensors for acquiring at least one parameter of the cardiopulmonary resuscitation according to the above description of the pad in conjunction with the apparatus according to the invention for cardiopulmonary resuscitation.

In embodiments of an apparatus according to the invention for cardiopulmonary resuscitation, the latter comprises a pad having such a guide device.

In embodiments of the method according to the invention for controlling an apparatus for cardiopulmonary resuscitation, use is made of a pad according to the invention in accordance with the description above.

Here, in preferred embodiments of the invention, a human aider is provided with information with regards to the direction and/or the distance that they should displace the position of the action of force along the search path for the purposes of optimizing the position of the action of force on the thorax of a patient during a cardiac massage.

The drawings illustrate exemplary embodiments of an apparatus according to the invention and a method according to the invention. In detail:

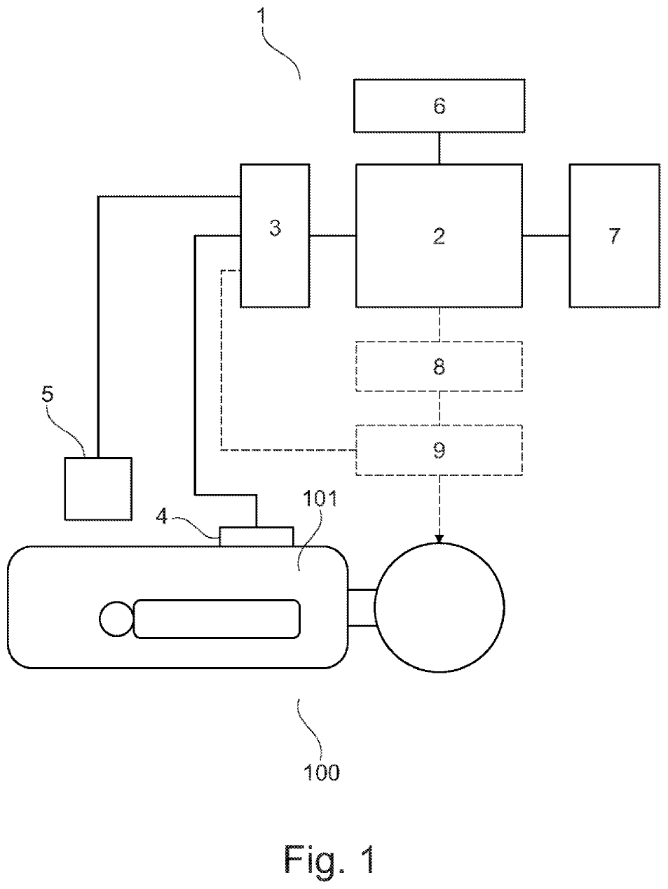

FIG. 1: shows a schematic representation of a block diagram of an embodiment of an apparatus according to the invention for cardiopulmonary resuscitation without a thorax compression device,

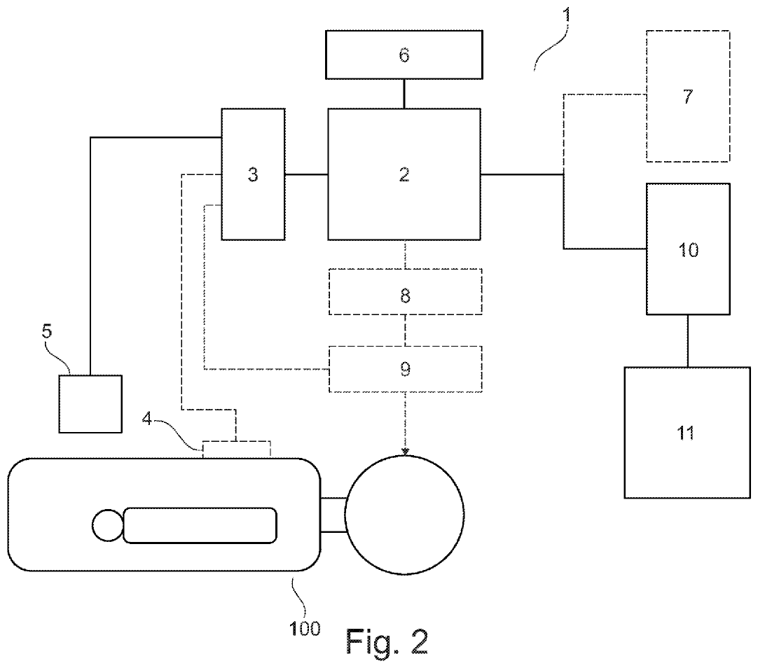

FIG. 2: shows a schematic representation of a block diagram of an embodiment of an apparatus according to the invention for cardiopulmonary resuscitation with a thorax compression device,

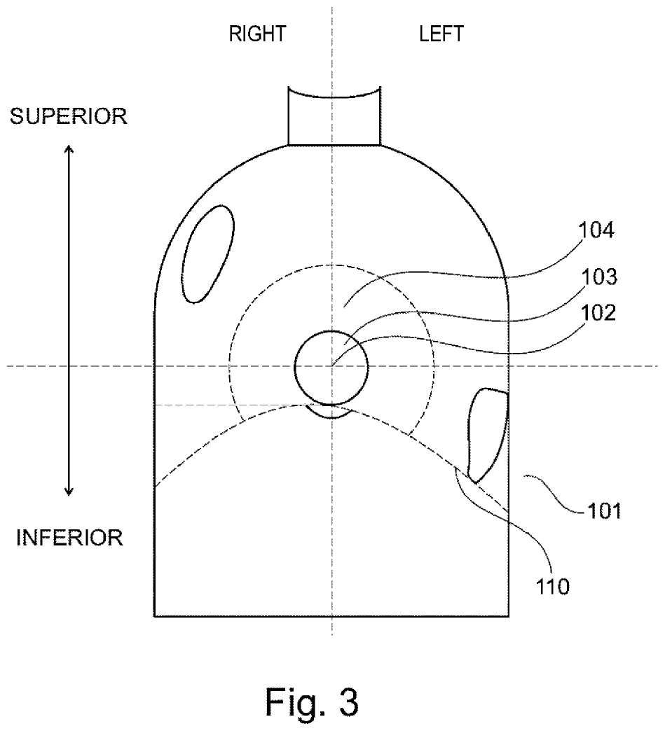

FIG. 3: shows a schematic representation of the torso of a patient in a frontal view,

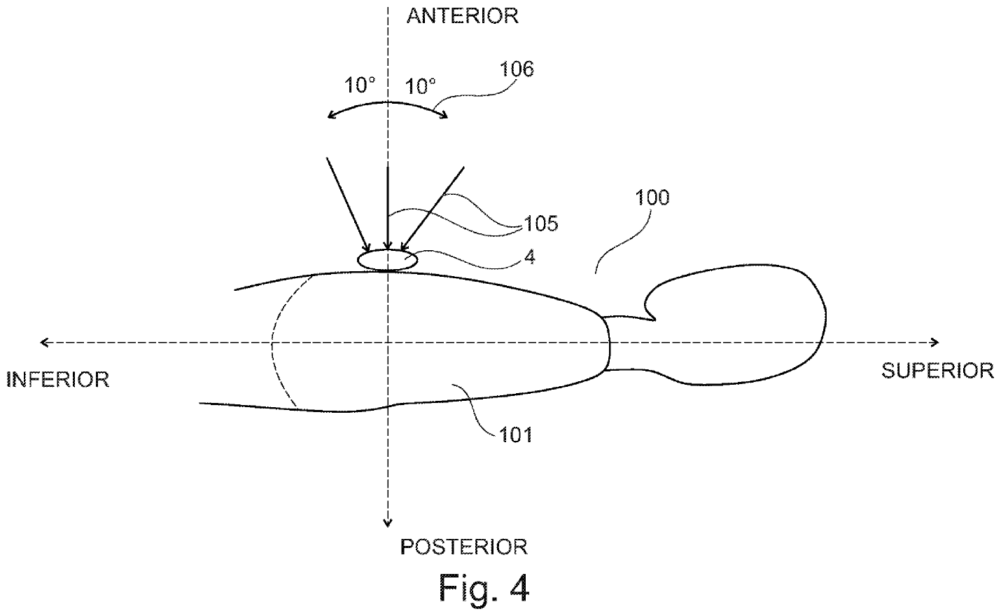

FIG. 4: shows a schematic representation of a section through the torso and head of a patient in the sagittal plane,

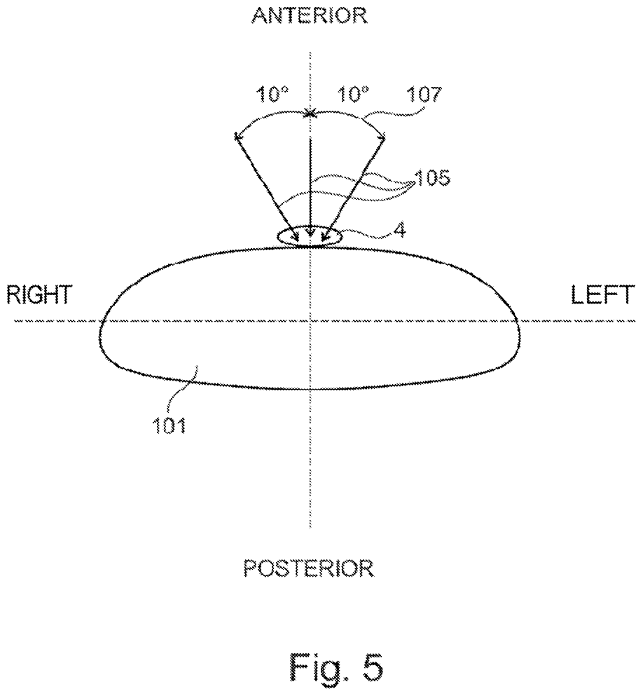

FIG. 5: shows a schematic representation of a section through the torso of a patient in the transverse plane,

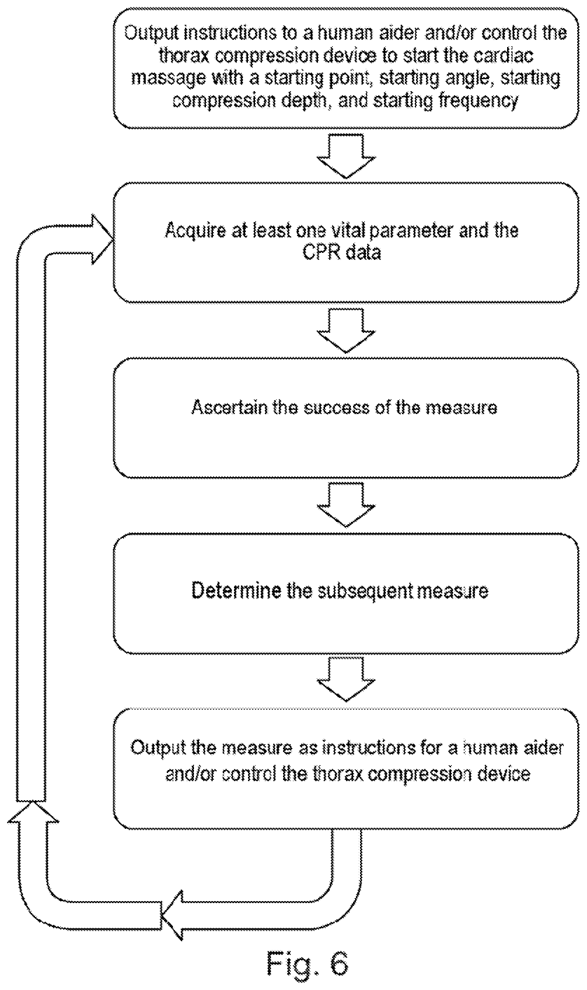

FIG. 6: shows an abstracted schematic representation of the progress of an embodiment of a method according to the invention for controlling an apparatus for cardiopulmonary resuscitation,

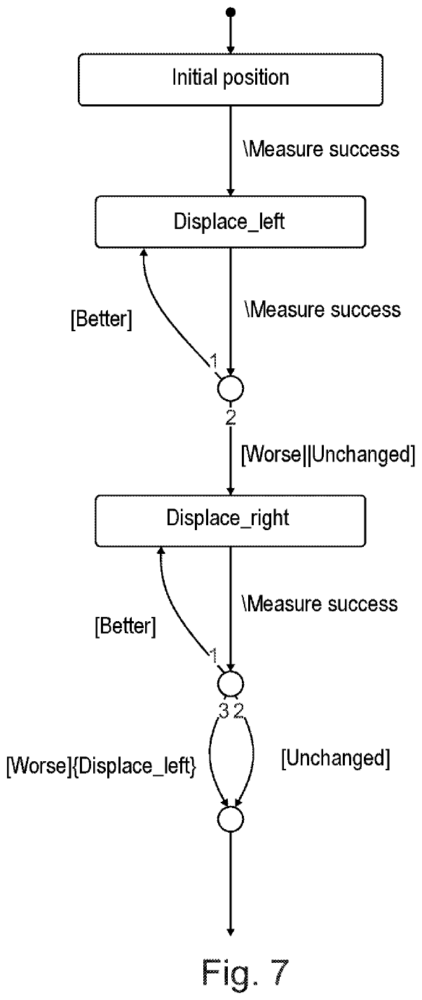

FIGS. 7 & 8: show a schematic representation of the progress of a component-by-component optimization of the position of the action of force on the thorax of a patient in an embodiment of a method according to the invention,

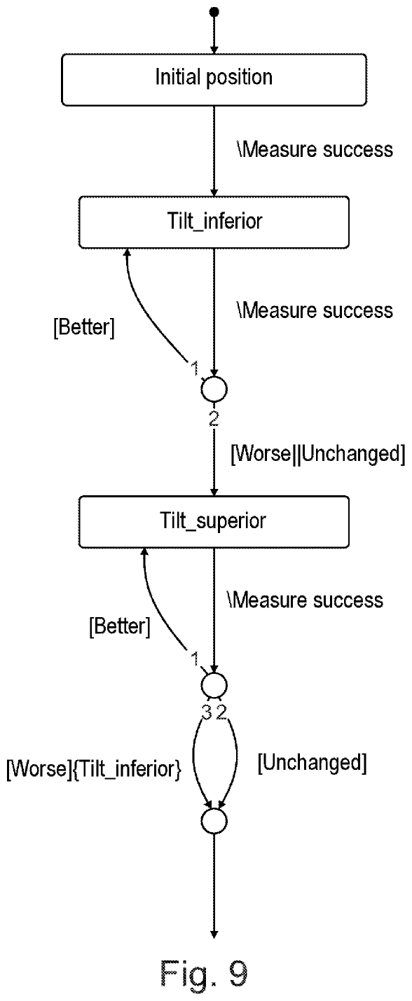

FIGS. 9 & 10: show a schematic representation of the progress of a component-by-component optimization of the direction of the action of force on the thorax of a patient in an embodiment of a method according to the invention,

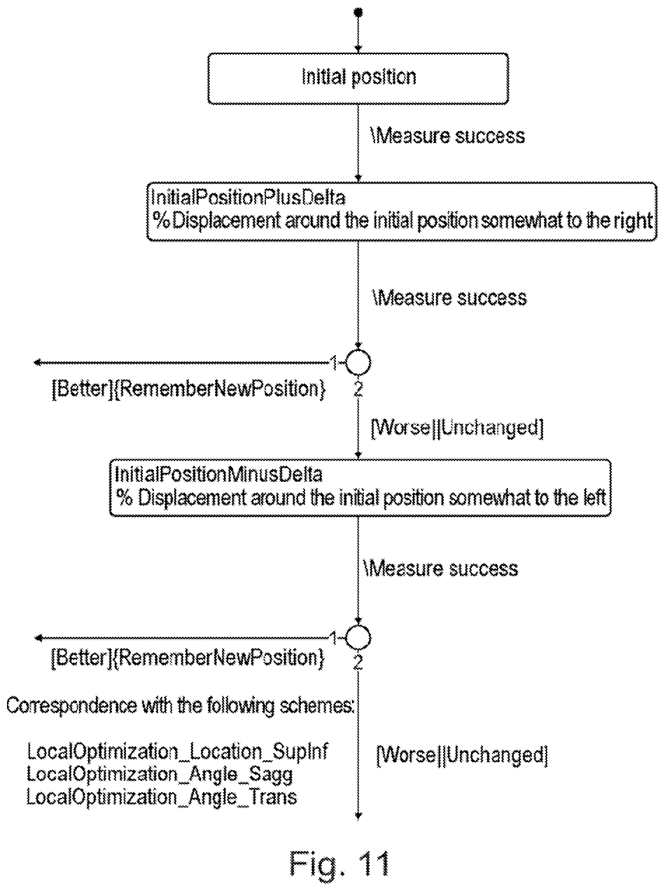

FIG. 11: shows a schematic representation of the progress of a continuous component-by-component fine alignment following the actual optimization of the parameters using the example of the transverse component of the position of the action of force on the thorax of a patient,

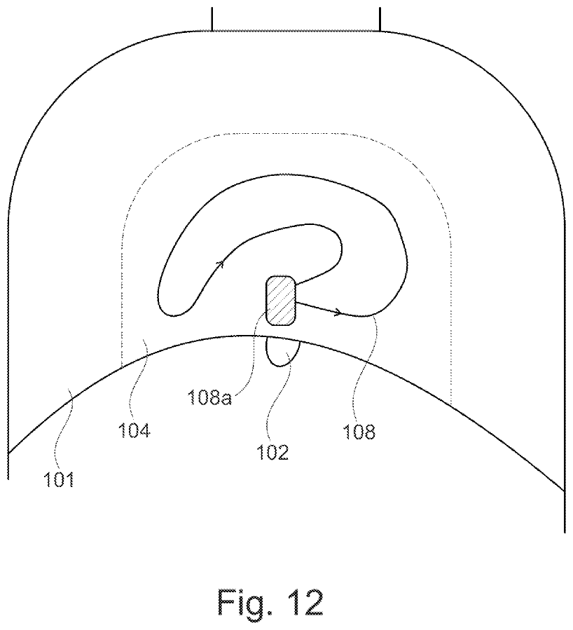

FIG. 12: shows a schematic representation of a search path in an embodiment of the method according to the invention,

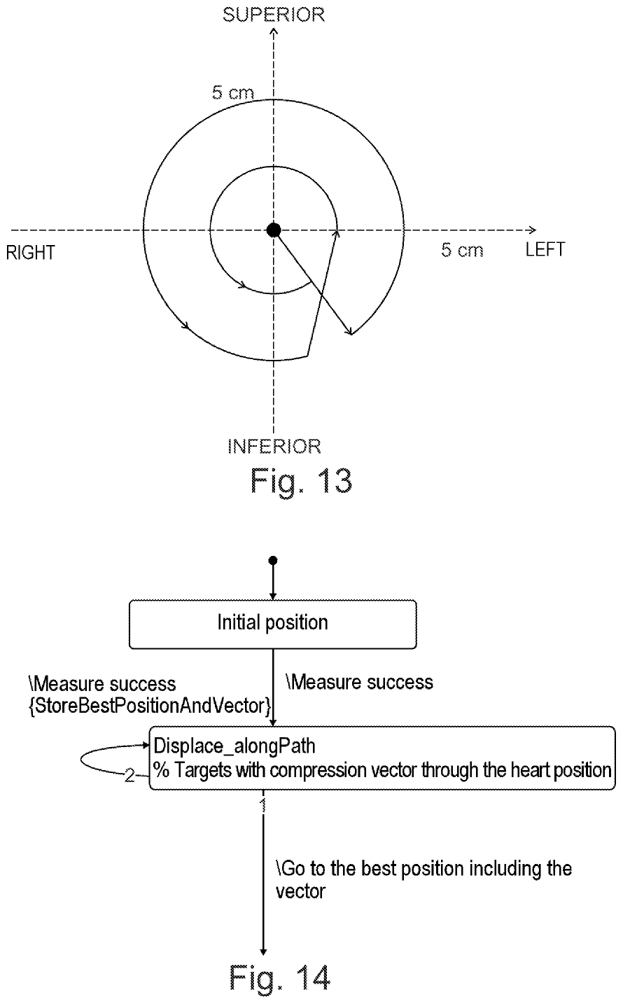

FIG. 13: shows a further schematic representation of a search path in an embodiment of the method according to the invention,

FIG. 14: shows a schematic representation of the progress of an optimization along a search path in an embodiment of a method according to the invention,

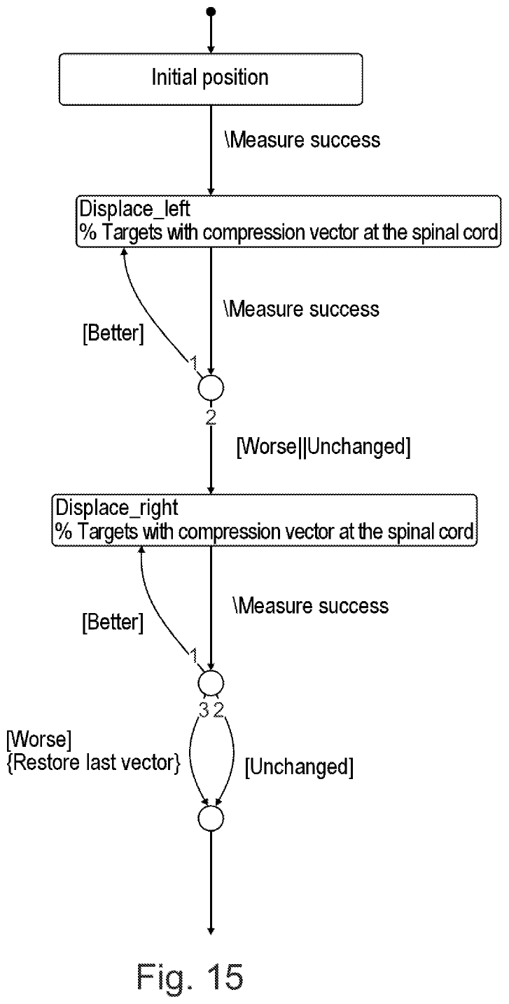

FIG. 15: shows a schematic representation of the progress of a vector optimization in an embodiment of a method according to the invention,

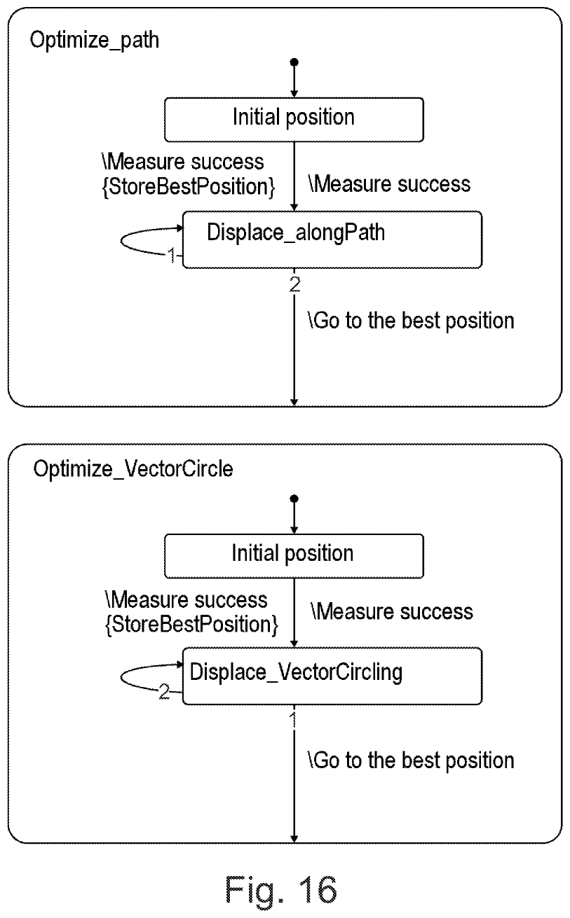

FIG. 16: shows a schematic representation of the progress of a combination of search path and vector optimization in an embodiment of a method according to the invention,

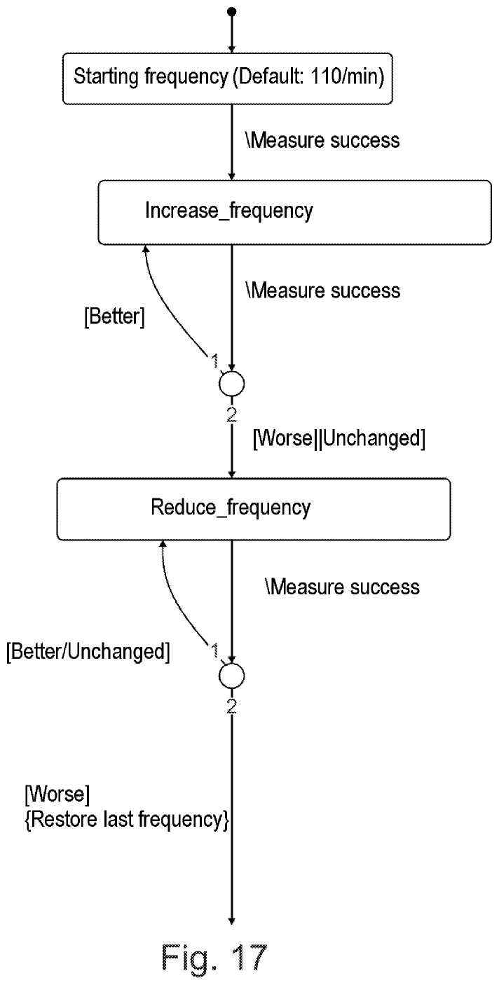

FIG. 17: shows a schematic representation of the progress of a frequency optimization in an embodiment of a method according to the invention, and

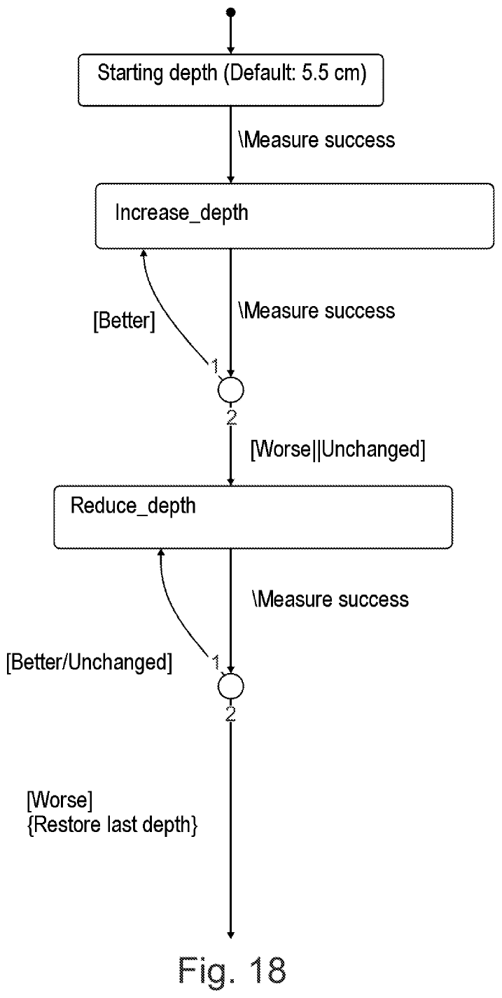

FIG. 18: shows a schematic representation of the progress of a compression depth optimization in an embodiment of a method according to the invention.

FIG. 1 schematically represents the block diagram of an embodiment according to the invention of an apparatus for cardiopulmonary resuscitation (1) without a thorax compression device (11).

The apparatus for cardiopulmonary resuscitation (1) comprises a computing unit (2), a sensor interface (3), a pad (4) for application or placement on the chest of a patient (100), at least one sensor (5) for acquiring at least one vital parameter, at least one memory (6), and an output device (7). An optional embodiment of an apparatus according to the invention for cardiopulmonary resuscitation (1) additionally comprising a control device for controlling the ventilation (8) and further optionally comprising a ventilator (9) is illustrated using dashed lines. The pad (4) comprises, either integrated therein or connected thereto in any other way, at least one acceleration sensor and/or gyroscope sensor. The sensors are connected to the sensor interface (3) such that the measured data acquirable with the aid of the sensors are transmittable to the computing unit (2) and/or the memory (6). Optical and/or acoustic instructions for a human aider with regard to carrying out a cardiac massage are able to be output with the aid of the output device (7).

With the aid of the illustrated embodiment of an apparatus according to the invention for cardiopulmonary resuscitation (1), at least one vital parameter corresponding to the implementation of a cardiac massage is rendered monitorable and the success of the cardiac massage over time is ascertainable therefrom with the aid of the computing unit (2). Instructions for adjusting at least one parameter of the cardiac massage are able to be output to a human aider with the aid of the output device (7) and the success of this measure is ascertainable by monitoring the at least one vital parameter with the aid of the computing unit (2).

FIG. 2 shows the block diagram of an embodiment according to the invention of an apparatus for cardiopulmonary resuscitation (1) comprising a thorax compression device (11). The thorax compression device (11) is designed so that the thorax (101) of a patient (100) is rendered compressible for the purpose of carrying out a cardiac massage. For the purpose of controlling the thorax compression device (11), the depicted embodiment of an apparatus for cardiopulmonary resuscitation (1) comprises a control unit (10). With the aid of the control unit (10), it is possible to generate control signals for controlling the thorax compression with the aid of the thorax compression device (11) and the said control signals are transmittable to the thorax compression device (11). In this embodiment of the invention, the output device (7) for outputting instructions to a human aider is optional. In an embodiment of the invention, the thorax compression device (11) is designed for the automated adjustment of all parameters of the cardiac massage. In another embodiment of the invention, the thorax compression device (11) relies on the operation by a human aider for the purpose of adjusting at least one parameter of the cardiac massage, for example the position of the action of force on the thorax (101) of a patient (100), with the output device (7) in this embodiment of the invention being designed to appropriately instruct the human aider. Moreover, the use of the pad (4) with the at least one integrated acceleration and/or gyroscope sensor for monitoring the compression depth and frequency parameters of the cardiac massage is optional in this embodiment of the invention. In embodiments of the invention where no pad (4) is used, the monitoring means for the compression depth and/or compression frequency of the cardiac massage is arranged in the region of the thorax compression device (11) or integrated in the latter.

FIG. 3 schematically illustrates the torso of a patient (100) in a frontal view. Depicted on the torso or the thorax (101) is the position (103) of the action of force on the thorax (101) of a patient (100) according to the guidelines, which is located in the lower region of the sternum (102), and the search region (104) according to the invention located around this position (103) for the purpose of ascertaining the optimal position of the action of force on the thorax (101) of a patient (100). Furthermore, the direction components of the position of the action of force on the thorax (101) of a patient (100) are depicted using right/left and superior/inferior. For instance, the search region (104) is delimited by the costal margin (110) in the inferior direction.

FIG. 4 shows a schematic representation of a section of the torso (101) and head of a patient (100) in the sagittal plane. The plotted coordinate axes define the superior direction in the direction of the head of the patient (100), inferior direction in the direction of the lower abdomen of the patient (100), anterior direction away from the chest of the patient (100) and posterior direction away from the back of the patient (100). A pad (4) has been placed on the upper side of the thorax (101) of the patient (100). A force is exerted onto the pad (4) for the purposes of carrying out a cardiac massage. The direction of the action of force on the pad (4) is given by the direction vector (105) of the action of force on the thorax (101) of the patient (100). According to the guidelines, the action of force is approximately perpendicular onto the pad (4). Within the scope of optimizing the direction of the action of force on the thorax (101) of the patient (100), the direction of the action of force on the thorax (101) is varied over a given first angular range (106) in the sagittal plane in an embodiment of a method according to the invention. In this case, the angle describes the degree of the tilt of the direction vector (105) of the action of force on the thorax (101) from the perpendicular direction according to the guidelines. In this case, the tilt of the direction vector (105) of the action of force can be implemented both in the inferior direction and in the superior direction. In the depicted embodiment of the invention, the first angular range (106) is realized by an angular range encompassing 20°, with the latter being divided symmetrically into a tilt of the direction vector (105) of 10° in the inferior direction or superior direction. In other embodiments of the invention, it is conceivable to have both different angular dimensions of the first angular range (106) and a non-symmetric division of the first angular range (106) in the inferior and superior directions.

FIG. 5 shows a schematic representation of a section of the torso (101) of a patient (100) in the transverse plane. The anterior direction, posterior direction and left and right directions are noted on the plotted coordinate axes. In accordance with the explanations regarding the variation of the direction vector (105) of the action of force on the thorax (101) in the sagittal plane, the direction vector (105) is tilted out of the perpendicular guideline-conform position within the scope of the optimization of the direction of the action of force on the thorax (101) of a patient (100) in an embodiment of a method according to the invention. In the transverse plane, the direction vector (105) is varied within a second angular range (107) which encompasses an angular range of 20° in the right or left directions. A symmetric distribution of the second angular range (107) of in each case 10° in the right and left direction is depicted. In other embodiments of the invention, other angular ranges of the second angular range (107) and/or non-symmetric distribution of the second angular range (107) among the left and right directions are conceivable.

FIG. 6 shows an abstracted schematic representation of the progress of an embodiment of a method according to the invention for controlling an apparatus for cardiopulmonary resuscitation (1). The depicted schematic progress comprises five method steps, with the individual method steps depicted in this figure each being able to be subdivided into further method steps. In the first method step, an instruction is output to a human aider and/or a thorax compression device (11) is controlled for the purpose of starting a cardiac massage at a starting point or a specific position of the action of force on the thorax (101) of a patient (100), a starting angle or direction vector (105) of the action of force on the thorax (101) which describes the direction of the action of force on the thorax (101), a starting compression depth, and a starting frequency of the cardiac massage.

At least one vital parameter of the patient (100) and the CPR data within the meaning of compression depth and the frequency of the cardiac massage of a cardiopulmonary resuscitation are acquired in the second method step.

Implemented abstractly in the third method step is the ascertainment of the success of the cardiac massage using the current parameters. If this method step is applied anew during the course of the method according to the invention, the success of the preceding measure is ascertained at this point.

In preferred embodiments of the invention, the method step of “ascertaining the success of the measure” comprises the readout of at least some of the stored previous measured data of the at least one vital parameter from a memory and the comparison of at least one measured value from a preceding measurement with at least one measured value of the current measurement.

The subsequent measure is determined in the fourth method step, with a measure being given by an adjustment of the instructions output to a human aider and/or of the control of a thorax compression device (11) in respect of the position of the action of force on the thorax (101), the direction of the action of force on the thorax (101), the compression depth and/or the frequency of the cardiac massage. The determination of the subsequent measure can in this case be based in embodiments of the method according to the invention on the ascertained success of a previous measure or on a specified sequence of measures.

In the fifth method step, the output of the determined measure is implemented as instructions for a human aider and/or by the corresponding control of a thorax compression device (11).

Subsequently, at least one vital parameter of the patient (100) and the CPR data are acquired anew in accordance with the second method step in the depicted embodiment of a method according to the invention, with a loop comprising method steps 2-5 being run through.

At least one termination criterion, the fulfillment of which terminates the loop, is defined in embodiments of the method according to the invention.

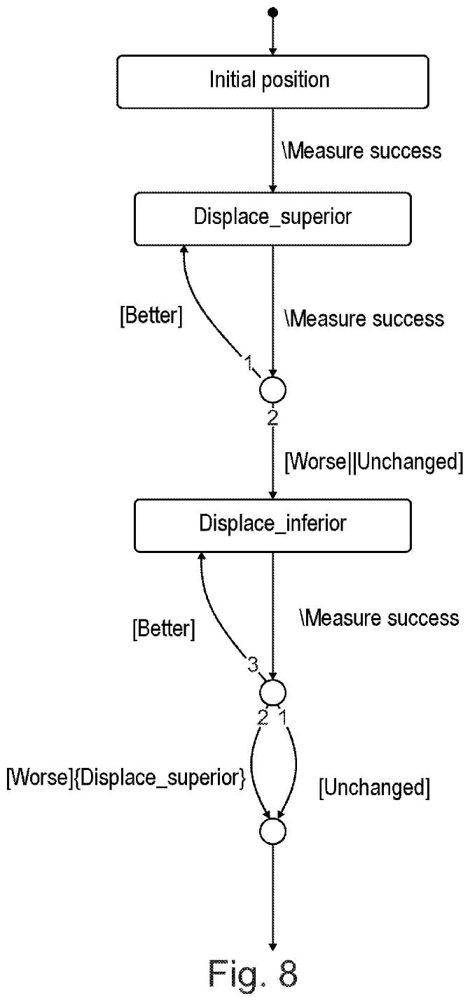

FIGS. 7 and 8 show a schematic representation of the progress of a component-by-component optimization of the position of the action of force on the thorax (101) of a patient (100) in an embodiment of a method according to the invention, with FIG. 7 representing the optimization of the right/left component of the position of the action of force and FIG. 8 representing the optimization of the position in the superior/inferior direction.

The following nomenclature is used in the figures relating to the optimization of individual parameters: if the term is preceded by a “/”, then this relates to the determination of a quantity; if the term is placed between square brackets (e.g., [better]), then this relates to an evaluation; and if the term is placed between curly brackets (e.g., {displace left}), then this relates to an action.

In both direction components, the optimization is implemented starting from an initial position, from which a displacement is carried out in one direction and the success of the measure provided by this displacement is subsequently determined. If an improvement has occurred in the process, then there is another displacement in the same direction, but if there is a deterioration or the state remains unchanged, then the position is displaced in the opposite direction. The success of the further measure provided by this displacement is subsequently determined. If an improvement in the state has occurred, then there is a renewed displacement in the same direction, but if the state has deteriorated, then there is a displacement in the opposite direction within the meaning of a measure. If the success of the measure is determined as unchanged, then there is no further displacement of the position in the respectively optimized direction and the optimization of the direction component has been completed.

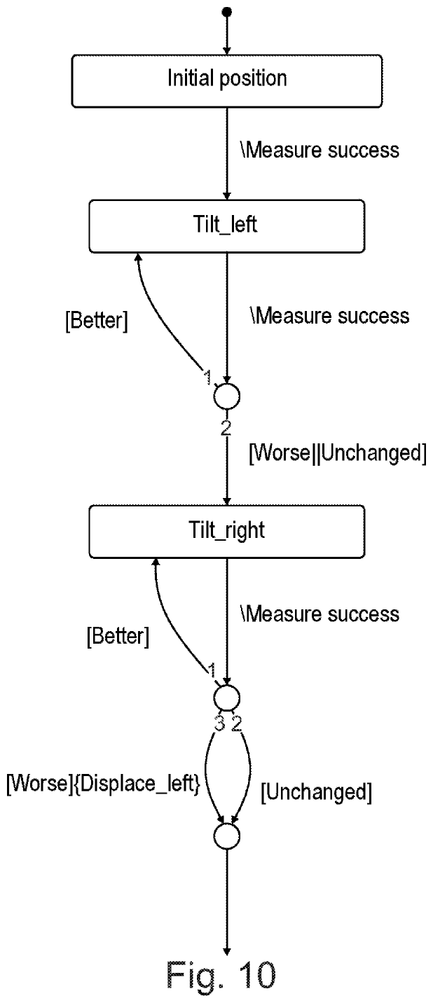

Analogously, FIGS. 9 and 10 show a schematic representation of the progress of a component-by-component optimization of the direction of the action of force on the thorax (101) of a patient (100) in an embodiment of a method according to the invention. In accordance with the displacement of the position of the action of force in the various directions, the measure here is however a tilt of the direction vector (105) of the action of force on the thorax (101) in the respective direction.

FIG. 11 represents the progress of a local optimization or re-optimization/readjustment of the position of the action of force on the thorax (101) using the example of the right/left direction component. Such a local optimization or readjustment follows an initial optimization of at least one parameter of the cardiac massage in embodiments of a method according to the invention. In this case, there is a displacement of the position of the action of force in a direction of the direction component, the determination of the success of this measure, and, as a determination of the subsequent measure, a definition of the new position provided an improvement has occurred or a displacement of the position in the opposite direction if a deterioration or no change has occurred. The flowcharts for optional local optimizations or re-optimizations of the superior/inferior direction component of the position and/or of the angular ranges in the sagittal or transverse plane may be designed analogously in embodiments of the invention.

FIG. 12 represents the optimization of at least the position of the action of force on the thorax (101) of a patient (100) on the basis of a search path optimization. In this case, the optimization of the position of the action of force is implemented along a predetermined search path (108). The optimization starts at a starting point (108a) of the search path (108). In this case, the search path (108) extends within a predefined search region (104). The displacement of the position of the action of force on the thorax (101) is in this case implemented incrementally along the search path (108), with the success of the measure given by the displacement along the search path (108) being ascertained continuously by acquiring and evaluating at least one vital parameter of the patient (100). Once the endpoint of the search path (108) has been reached, the said endpoint also corresponding to the starting point (108a) in the depicted embodiment of the method according to the invention, the position of the action of force on the thorax (101) where the greatest success was ascertained is determined.

This optimized position is output as an instruction to a human aider and/or used to control a thorax compression device (11).

FIG. 13 shows a schematic representation of the projection of the search vector (109) in a frontal view of the thorax (101). From the starting point (108a), there is in this case a displacement of the position of the action of force on the thorax (101) in a range of +/−5 cm, both in the right/left direction component and in the superior/inferior direction component. The search vector (109) specifies the direction of the positional change along a search path (108).

FIG. 14 schematically represents the progress of a vector path optimization of an embodiment of a method according to the invention. There is within the scope of vector path optimization a displacement along a search path (108) of the position of the action of force on the thorax (101), with the direction vector of the direction of the action of force on the thorax (101) being aligned in such a way at each position on the search path (108) that the said direction vector is directed at the position of the heart of the patient (100). In accordance with the progresses of the optimization illustrated in the previous figures, the vector path optimization also includes a displacement of the position along the search path (108) and additionally an adjustment of the direction vector (105) of the action of force within the meaning of a measure of a method according to the invention. Once the search path (108) has been traversed, there is a comparison of the successes determined for the different positions and the corresponding direction vectors and the selection of the position with the greatest success.

FIG. 15 shows a flowchart for the combined optimization of vector and position in the individual direction components of the position of the action of force on the thorax (101).

FIG. 16 illustrates the path/vector optimization with predefined search path (108) and search vector (109).

FIG. 17 shows the frequency optimization and FIG. 18 shows the compression depth optimization of an embodiment according to the invention of a method for controlling an apparatus for cardiopulmonary resuscitation (1).

1-18. (canceled)

19. An apparatus for cardiopulmonary resuscitation, comprising: at least one computing unit; at least one sensor interface for establishing a connection to at least one sensor; and a memory, wherein at least one vital parameter of a patient is rendered acquirable by the at least one sensor, wherein the acquired measured values of the at least one vital parameter are storable in the memory, wherein the computing unit is configured to ascertain success of a cardiac massage, and wherein a measure for adjusting at least one parameter of the cardiac massage is derivable from the ascertained success.

20. The apparatus for cardiopulmonary resuscitation according to claim 19, further comprising a ventilation apparatus for ventilating the patient and a control device for controlling the ventilation.

21. The apparatus for cardiopulmonary resuscitation according to claim 19, further comprising an output unit for the optical and/or acoustic output of instructions for a human aider.

22. The apparatus for cardiopulmonary resuscitation according to claim 19, further comprising a thorax compression device for compressing the thorax of the patient and a control device for controlling the thorax compression device.

23. The apparatus for cardiopulmonary resuscitation according to claim 19, further comprising a thorax compression device and an output device for outputting instructions to a human aider.

24. The apparatus for cardiopulmonary resuscitation according to claim 22, wherein the thorax compression device is configured so that a position of an action of force on the thorax of the patient and/or a direction of the action of force on the thorax of the patient is modifiable based on a measure that is determinable by the computing unit.

25. A pad for use with an apparatus for cardiopulmonary resuscitation apparatus according to claim 19, comprising a guide device that defines a search path, a thorax of a patient being compressed along the search path within the scope of a cardiopulmonary resuscitation.

26. The pad according to claim 25, wherein the guide device is realized by an optical marking on the pad or a haptic design of a surface of the pad.

27. The pad according to claim 25, wherein the guide device is a guide rail that extends along the search path and in which a handle is displaceably mounted so that an action of force on the handle at a respective position along the search path is transferred to the thorax of the patient.

28. A method for controlling an apparatus for cardiopulmonary resuscitation, comprising the steps of: outputting an instruction to a human aider and/or controlling a thorax compression device for purposes of starting a cardiac massage with starting parameters including a starting position, a starting angle, a starting compression depth, and a starting frequency; acquiring at least one vital parameter of a patient and the compression depth and the frequency of the cardiac massage; ascertaining success of the cardiac massage using the starting parameters; determining a measure that corresponds to an adjustment of at least one of the parameters of a position of an action of force, direction of the action of force, compression depth and frequency of the cardiac massage; and outputting a previously determined measure as an instruction to the human aider controlling the thorax compression device.

29. The method for controlling an apparatus for cardiopulmonary resuscitation according to claim 28, wherein the outputting of the previously determined measure to a human aider and/or the controlling of a thorax compression device is followed by an acquisition of at least one vital parameter of the patient, and a success of the measure is ascertained in a further method step, and a determination of a subsequent measure is implemented in a further method step.

30. The method for controlling an apparatus for cardiopulmonary resuscitation according to claim 29, including implementing the determination of the subsequent measure based on the success of the preceding measure.

31. The method for controlling an apparatus for cardiopulmonary resuscitation according to claim 28, including optimizing at least one of the parameters of the position of the action of force on the thorax of the patient, the direction of the action of force on the thorax of the patient, the compression depth, and the frequency of the cardiac massage by determining the measures and ascertaining the success of the respective measure.

32. The method for controlling an apparatus for cardiopulmonary resuscitation according to claim 31, including implementing the optimization of the position of the action of force on the thorax of the patient and/or the optimization of the direction of the action of force on the thorax of the patient is implemented along a search path, with a traversal of the search path being followed by ascertainment of the position and/or direction of the action of force on the thorax of the patient where success was greatest.

33. The method for controlling an apparatus for cardiopulmonary resuscitation according to claim 32, wherein the search path is specified and loaded from a memory at a start of the method, with the subsequent measure being given by a next point on the search path, at least during the optimization of the parameters of the position of the action of force on the thorax of the patient or a joint optimization of the position and direction of the action of force on the thorax of the patient.

34. The method for controlling an apparatus for cardiopulmonary resuscitation according to claim 32, including optimizing the direction of the action of force on the thorax of the patient at each point along the search path based on an optimization process and ascertaining an interaction of position and direction of the action of force on the thorax of the patient with the greatest success following the traversal of the search path.

35. The method for controlling an apparatus for cardiopulmonary resuscitation according to claim 31, including a component-by-component optimization of the parameters of position and direction of the action of force on the thorax of a patient.

36. The method for controlling an apparatus for cardiopulmonary resuscitation according to claim 28, including using an apparatus for cardiopulmonary resuscitation that comprises: at least one computing unit; at least one sensor interface for establishing a connection to at least one sensor; and a memory, wherein at least one vital parameter of a patient is rendered acquirable by the at least one sensor, wherein the acquired measured values of the at least one vital parameter are storable in the memory, wherein the computing unit is configured to ascertain success of a cardiac massage, and wherein a measure for adjusting at least one parameter of the cardiac massage is derivable from the ascertained success and/or using a pad that comprises a guide device that defines a search path, a thorax of a patient being compressed along the search path within the scope of a cardiopulmonary resuscitation.
